(19) United States
(12) Patent Application Publication
(10) Pub. No.: US 2008/0206173 A1
(43) Pub. Date: Aug. 28, 2008

(54) NAIL COMPOSITIONS IN POWDERED FORM

(75) Inventors: Robert WEBER, Suffern, NY (US); Nishith Patel, Roselle Park, NJ (US)

Correspondence Address: L'OREAL USA/ PATENT DEPARTMENT, 30 TERMINAL AVENUE, CLARK, NJ, 07066, US

(73) Assignee: L'OREAL, Paris (FR)

(21) Appl. No.: 12/035,183

(22) Filed: Feb. 21, 2008

Related U.S. Application Data
(60) Provisional application No. 60/903,234, filed on Feb. 23, 2007.
(60) Provisional application No. 60/906,607, filed on Mar. 13, 2007.

(51) Int. Cl. A61K 8/72; A61Q 3/02

(52) U.S. Cl. 424/61

(57) ABSTRACT

A process for making up nails involving the steps of: (a) providing a first composition, in powder form, containing: (i) at least one colorant, in powder form; (ii) optionally, at least one film-forming polymer, in powder form; and (iii) optionally, at least one plasticizer; (b) providing a second composition containing: (iv) at least one solvent; (v) at least one film-forming polymer chosen from nitrocellulose, cellulose acetate butyrate, and mixtures thereof; and (vi) at least one plasticizer; (c) depositing the first composition onto a nail surface; and (d) applying the second composition over-top of the first composition.

This application is based on and claims the benefit of U.S. Provisional Application Ser. No. 60/903,234, entitled NAIL COMPOSITIONS IN POWDERED FORM, filed Feb. 23, 2007, and of U.S. Provisional Application Ser. No. 60/906,607, entitled NAIL COMPOSITIONS IN POWDERED FORM, filed Mar. 13, 2007, the entire disclosures of which are incorporated by reference herein.

### BACKGROUND OF THE INVENTION

Nail polish varnishes are typically sold to, and applied by, consumers as ready-to-use formulations. As a result, caution must be exercised when using the product in order to prevent inadvertent deposition of the nail varnish onto surfaces other than the nail.

A need, therefore, exists for a nail polish composition and system which reduces the likelihood of unwanted application of a nail polish varnish onto surfaces other than the nail.

### SUMMARY OF THE INVENTION

The present invention is directed to a process for making up nails involving the steps of: (a) providing a first composition, in powder form, containing: (i) at least one colorant, in powder form; (ii) optionally, at least one film-forming polymer, in powder form; and (iii) optionally, at least one plasticizer; (b) providing a second composition containing: (iv) at least one solvent; (v) at least one film-forming polymer chosen from nitrocellulose, cellulose acetate butyrate, and mixtures thereof; and (vi) at least one plasticizer; (c) depositing the first composition onto a nail surface; and (d) applying the second composition over-top of the first composition.

Another aspect of the present invention relates to a cosmetic kit for making up nails comprising a multi-unit receptacle containing: (a) at least one unit having a powder-form composition therein, the composition containing: (i) at least one colorant, in powder form; (ii) optionally, at least one film-forming polymer, in powder form; and (iii) optionally, at least one plasticizer; (b) at least one unit having a liquid-form composition therein, the composition containing: (iv) at least one solvent; (v) at least one film-forming polymer chosen from nitrocellulose, cellulose acetate butyrate, and mixtures thereof; and (vi) at least one plasticizer; (c) at least one tool capable of depositing the powder-form composition onto the nail; and (d) at least one tool capable of applying the liquid-form composition over-top of the powder-form composition.

### DETAILED DESCRIPTION OF THE INVENTION

Other than in the operating examples, or where otherwise indicated, all numbers expressing quantities of ingredients and/or reaction conditions are to be understood as being modified in all instances by the term “about”.

First Powder-Form Composition

The first composition, in powder form, contains at least one colorant, in powder form; optionally, at least one film-forming polymer, in powder form; and optionally, at least one plasticizer.

Colorant

Suitable colorants for use in the first composition include pulverulent compounds and/or dyes which are soluble in the solvent of the composition.

The pulverulent compounds can be chosen from pigments and/or pearlescent agents and/or glitter generally used in nail varnishes.

The pigments can be white or colored and inorganic and/or organic. Mention may be made, among inorganic pigments, of titanium dioxide, which has optionally been surface-treated, zirconium, cerium, iron or chromium oxides, manganese violet, ultramarine blue, chromium hydrate, ferric blue or metal pigments, such as aluminum, copper or bronze. Mention may be made, among organic pigments, of carbon black, pigments of D & C type, lakes based on cochineal carmine or on barium, strontium, calcium or aluminum, or guanine.

Other pigments may also be chosen from pigments with an effect, such as particles comprising an organic or inorganic and natural or synthetic substrate, for example glass, acrylic, polyester, polyurethane or poly(ethylene terephthalate) resins, ceramics or aluminas, which substrate may or may not be covered with metal substances, such as aluminum, gold, copper or bronze, or with metal oxides, such as titanium dioxide, iron oxide or chromium oxide, or with inorganic or organic pigments, and their mixtures.

The pearlescent pigments may be chosen from white pearlescent pigments, such as mica covered with titanium oxide or with bismuth oxychloride, colored pearlescent pigments, such as titanium oxide-coated mica with iron oxides, titanium oxide-coated mica with in particular ferric blue or chromium oxide, or titanium oxide-coated mica with an organic pigment of the abovementioned type, and fluorophlogopites with iron oxides.

Other pigments may include those with goniochromatic properties, in particular liquid crystal or multilayer pigments.

The dyes are, for example, Sudan red, DC Red 17, DC Green 6, β-carotene, soybean oil, Sudan brown, DC Yellow 11, DC Violet 2, DC Orange 5 or quinoline yellow.

The colorant may also be chosen from fluorescent whitening agents.

In a preferred embodiment, the colorant is employed in an amount sufficient to enable single coat coverage of the nail. The precise amount used depends on the color, shade and style of nail polish varnish being formulated and, as a result, will be determined by those of ordinary skill in the art.

In general, the colorant will be present in an amount of from about 1 to about 100% by weight, preferably from about 90 to about 100% by weight, based on the total weight of the first powder-form composition.

Film-Forming Polymer

The term “film-forming polymer” is understood to mean a polymer capable of forming, alone and/or in the presence of a plasticizer, an isolable film.

The film-forming polymer can be chosen from radical polymers, polycondensates and polymers of natural origin.

Suitable film-forming polymers include, but are not limited to, vinyl polymers such as, for example, polyvinyl butyral, acrylic (co)polymers, polyurethanes, polyesters, alkyd resins, epoxy ester resins, cellulose polymers, such as nitrocellulose, cellulose esters, such as cellulose acetate, cellulose acetate propionate or cellulose acetate butyrate, resins resulting from the condensation of formaldehyde with an arylsulphonamide, and their mixtures. Other suitable film forming polymers may also include film formers which are more compatible with water. Such film formers include, but are not limited to starches and derivatives thereof, natural or synthetic gums and derivatives thereof, water soluble adhesives. One example of a water soluble adhesive includes but is not limited to, Gantrez S, a polymer containing the repeating unit of Formula I:

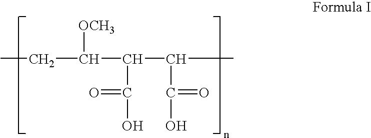

which is commercially available from the ISP corporation.

The film-forming polymer may be present in the first powder-form composition in an amount of up to about 30% by weight, such as ranging from about 0.1% to about 30% by weight, such as from about 5% to about 30% by weight, based on the total weight of the first composition.

The first powder-form composition may additionally comprise at least one plasticizer. Suitable plasticizers include, but are not limited to, glycols and their ether or ester derivatives, esters of acids, in particular carboxylic acids, such as citrates, adipates, carbonates, tartrates, phosphates or sebacates, oxyethylenated derivatives, such as oxyethylenated oils, and their mixtures.

The plasticizer may be present in the first composition according to the invention in an amount of up to about 20% by weight, such as ranging from about 0.1% to about 20% by weight, such as from about 1% to about 20% by weight, based on the total weight of the first composition.

It should be noted that in order to avoid a gritty, aesthetically unappealing, application of the first powder-form composition onto the nail, both the colorant and film-forming polymer should have substantially the same particle size distribution.

Second Liquid-Form Composition

The second composition, in liquid form, contains at least one solvent; at least one film-forming polymer chosen from nitrocellulose, cellulose acetate butyrate, and mixtures thereof; and at least one plasticizer. The second liquid form composition acts as a top coat, effectively sealing the first powder coat composition onto the nail surface.

Solvent

Suitable organic solvents are those which are liquid at ambient temperature and include, but are not limited to,:

ketones such as methyl ethyl ketone, methyl isobutyl ketone, diisobutyl ketone, isophorone, cyclohexanone or acetone;

alcohols, such as ethanol, isopropanol, diacetone alcohol, 2-butoxyethanol or cyclohexanol;

glycols, such as ethylene glycol, propylene glycol, pentylene glycol or glycerol;

propylene glycol ethers, such as propylene glycol monomethyl ether, propylene glycol monomethyl ether acetate or dipropylene glycol mono(n-butyl) ether;

short-chain esters (having a total of 2 to 7 carbon atoms), such as ethyl acetate, methyl acetate, propyl acetate, n-butyl acetate or isopentyl acetate;

alkanes, such as decane, heptane, dodecane or cyclohexane;

aldehydes, such as benzaldehyde or acetaldehyde;

water and

their mixtures.

The organic solvent is preferably chosen from short-chain esters (having a total of from 2 to 8 carbon atoms) alcohols, and their mixtures.

The at least one solvent is present in an amount of from about 30 to about 90% by weight, based on the weight of the second composition.

It should be understood that, in the event a plasticizer and/or a film former are present in the first powder-form composition, the solvent in the second liquid-form composition is capable of dissolving such ingredients.

It should also be understood that the solvent in the second liquid-form composition is capable of dissolving the film formers and plasticizers that are present in said composition.

The film-forming polymer for use in the second liquid-form composition of the present invention is chosen from nitrocellulose, cellulose acetate butyrate, and mixtures thereof, as well as from film formers which are more compatible with water. Those include but are not limited to latex, polyurethanes, acrylics and the like, such as Styrene/Acrylates copolymer.

The film-forming polymer is present in an amount of from about 5 to about 70% by weight, such as from about 10 to about 60% by weight, such as from about 15 to about 50% by weight, based on the weight of the second composition.

The plasticizer used in the second liquid-form composition of the present invention may be chosen from the previously-disclosed list of plasticizer candidates.

The plasticizer is present in an amount of from about 1 to about 20% by weight, such as from about 2 to about 15% by weight, such as from about 3 to 12%, based on the weight of the second composition.

Water Repelling Agents

The second liquid-form composition may also comprise a water repelling agent, which imparts water resistance to the top coat. Suitable water repelling agents are those which are compatible with conventional nail enamel solvents and resins. They include, but are not limited to, silicone oils such as dimethicone. Water repelling agents may be present in the composition of the present invention in an amount of from about 0.01% to about 10% by weight, based on the weight of the second composition.

Auxiliaries/Additives

The composition may additionally comprise an additive or auxiliary commonly used in cosmetic compositions and known to a person skilled in the art as being capable of being incorporated into a nail varnish composition.

Such additives or auxiliaries may be chosen from thickeners, coalescents, preservatives, fragrances, oils, waxes, surfactants, antioxidants, agents for combating free radicals, spreading agents, wetting agents, dispersing agents, antifoaming agents, neutralizing agents, stabilizing agents, active principles chosen from essential oils, UV screening agents, sunscreens, moisturizing agents, vitamins, proteins, ceramides, plant extracts, fibers, and the like, and their mixtures.

Of course, a person skilled in the art will take care to choose these possible additional compounds and/or their amounts so that the properties of the composition according to the invention are not, or not substantially, detrimentally affected by the envisaged addition.

In operation, the process for making up nails involves depositing the first powder-form composition onto a nail surface and then applying the second liquid-form composition over-top of the first composition.

Another aspect of the present invention relates to a cosmetic kit for making up nails comprising a multi-unit receptacle containing: (a) at least one unit having a powder-form composition therein, the composition containing: (i) at least one colorant, in powder form; (ii) optionally, at least one film-forming polymer, in powder form; and (iii) optionally, at least one plasticizer; (b) at least one unit having a liquid-form composition therein, the composition containing: (iv) at least one solvent; (v) at least one film-forming polymer chosen from nitrocellulose, cellulose acetate butyrate, and mixtures thereof; and (vi) at least one plasticizer; (c) at least one tool capable of depositing the powder-form composition onto the nail; and (d) at least one tool capable of applying the liquid-form composition over-top of the powder-form composition.

The tool used for depositing the first powder-form composition onto the nail should be of the type capable of carrying powder-form ingredients. The tool used for applying the second liquid-form composition over-top of the first composition is known to those skilled in the art such as, for example, a small artist brush. It should be understood, however, that any means suitable for effectuating the processes of the present invention may be employed without departing from the spirit of the present invention.

The present invention will be better understood from the examples which follow, all of which are intended for illustrative purposes only, and are not meant to unduly limit the scope of the invention in any way.

### EXAMPLES

First Powder-Form Compositions

### Examples 1-3

| | Ex. 1 | Ex. 2 | Ex. 3 |
|---|---|---|---|
| Ingredients | W/W % | W/W % | W/W % |
| Xirona Le Rouge | 89.95 | 0 | 0 |
| Suncroma D & C Red 6 | 3.50 | 0 | 0 |
| Acetyl Tributyl Citrate | 6.55 | 13.82 | 0 |
| TiO2 & Oxidized Polyethylene | 0 | 30.90 | 0 |
| Prestige soft beige | 0 | 37.64 | 0 |
| KTZ Fine white | 0 | 17.64 | 0 |
| Cloisonne satin rouge | 0 | 0 | 57.14 |
| Flamenco satin violet | 0 | 0 | 45.86 |
| Total | 100.00 | 100.00 | 100.00 |

Second Liquid-Form Composition

### Example 4

| | Ex. 4 |
|---|---|
| Ingredients | W/W % |
| Nitrocellulose | 11.1 |
| Ethyl Tosylamide | 2.8 |
| Epoxy Resin | 2.1 |
| Isopropyl Alcohol | 11.1 |
| Sucrose Acetate Isobutyrate | 3.4 |
| Sucrose Benzoate | 3.2 |
| Propyl Acetate | 9.1 |
| Butyl Acetate | 29.5 |
| Ethyl Acetate | 27.2 |
| Dimethicone | 0.5 |
| Total | 100.0 |

Additional Water Based Examples

First Powder Form Composition

### Example 5

| | Ex. 5 |
|---|---|
| Ingredients | W/W % |
| Xirona Le Rouge | 92.50 |
| Suncroma D & C Red 6 | 3.50 |
| Gantrez S | 3.00 |
| PROPYLENE GLYCOL | 1.00 |
| Total | 100.00 |

Second Liquid-Form Composition

### Example 6

| Ingredients | Ex. 6 |
|---|---|
| Trimethyl Hydroxypentyl Isobutyrate | 1.0 |
| Sodium Magnesium Silicate | 1.0 |
| Styrene/Acrylates Copolymer | 29.0 |
| Phenoxyethanol | 0.5 |
| PEG-12 Dimethicone | 1.5 |
| Water | 63.0 |
| PPG-2 Methyl Ether | 3.5 |
| Propylene Glycol | 0.5 |
| Total | 100.0 |

Once the second liquid form composition has dried on the nail, the user can rinse his/her hands in cool running water to remove any excess powder applied to the surrounding area of the nail.

What is claimed is:

1. A process for making up nails comprising:
(a) providing a first composition, in powder form, containing:
(i) at least one colorant, in powder form;
(ii) optionally, at least one film-forming polymer, in powder form; and
(iii) optionally, at least one plasticizer;
(b) providing a second composition containing:
(iv) at least one solvent;
(v) at least one film-forming polymer chosen from nitrocellulose, cellulose acetate butyrate, latex, polyurethanes, acrylics, and mixtures thereof; and
(vi) at least one plasticizer;
(c) depositing the first composition onto a nail surface; and
(d) applying the second composition over-top of the first composition.

2. The process of claim 1 wherein (a) (i) is present in an amount of from about 1 to about 100% by weight, based on the weight of the first composition.

3. The process of claim 1 wherein (a) (ii) is present in an amount of from about 5 to about 30% by weight, based on the weight of the first composition.

4. The process of claim 1 wherein (a) (iii) is present in an amount of from about 1 to about 20% by weight, based on the weight of the first composition.

5. The process of claim 1 wherein (a) (i) and (a) (ii) have substantially equal particle size distributions.

6. The process of claim 1 wherein (b) (iv) is present in an amount of from about 30 to about 90% by weight, based on the weight of the second composition.

7. The process of claim 1 wherein (b) (iv) is capable of dissolving (a)(ii) and/or (a)(iii).

8. The process of claim 1 wherein (b) (v) is present in an amount of from about 15 to about 50% by weight, based on the weight of the second composition.

9. The process of claim 1 wherein (b) (vi) is present in an amount of from about 3 to about 12% by weight, based on the weight of the second composition.

10. The process of claim 1 wherein (a) (i) is present in an amount of from about 1 to about 100% by weight, based on the weight of the first composition, (a) (ii) is present in an amount of from about 5 to about 30% by weight, based on the weight of the first composition, (a) (iii) is present in an amount of from about 1 to about 20% by weight, based on the weight of the first composition, (b) (iv) is present in an amount of from about 30 to about 90% by weight, based on the weight of the second composition, (b) (v) is present in an amount of from about 15 to about 50% by weight, based on the weight of the second composition, and (b) (vi) is present in an amount of from about 3 to about 12% by weight, based on the weight of the second composition.

11. A cosmetic kit for making up nails comprising a multi-unit receptacle containing:
(a) at least one unit having a powder-form composition therein, the composition containing:
(i) at least one colorant, in powder form;
(ii) optionally, at least one film-forming polymer, in powder form; and
(iii) optionally, at least one plasticizer;
(b) at least one unit having a liquid-form composition therein, the composition containing:
(iv) at least one solvent;
(v) at least one film-forming polymer chosen from nitrocellulose, cellulose acetate butyrate, latex, polyurethanes, acrylics, and mixtures thereof; and
(vi) at least one plasticizer;
(c) at least one tool capable of depositing the powder-form composition onto the nail; and
(d) at least one tool capable of applying the liquid-form composition over-top of the powder-form composition.

12. The kit of claim 11 wherein (a) (i) is present in an amount of from about 1 to about 100% by weight, based on the weight of the first composition.

13. The kit of claim 11 wherein (a) (ii) is present in an amount of from about 5 to about 30% by weight, based on the weight of the first composition.

14. The kit of claim 11 wherein (a) (iii) is present in an amount of from about 1 to about 20% by weight, based on the weight of the first composition.

15. The kit of claim 11 wherein (a) (i) and (a) (ii) have substantially equal particle size distributions.

16. The kit of claim 11 wherein (b) (iv) is present in an amount of from about 30 to about 90% by weight, based on the weight of the second composition.

17. The kit of claim 11 wherein (b) (iv) is capable of dissolving (a)(ii) and/or (a)(iii).

18. The kit of claim 11 wherein (b) (v) is present in an amount of from about 15 to about 50% by weight, based on the weight of the second composition.

19. The kit of claim 11 wherein (b) (vi) is present in an amount of from about 3 to about 12% by weight, based on the weight of the second composition.

20. The kit of claim 11 wherein (a) (i) is present in an amount of from about 1 to about 100% by weight, based on the weight of the first composition, (a) (ii) is present in an amount of from about 5 to about 30% by weight, based on the weight of the first composition, (a) (iii) is present in an amount of from about 1 to about 20% by weight, based on the weight of the first composition, (b) (iv) is present in an amount of from about 30 to about 90% by weight, based on the weight of the second composition, (b) (v) is present in an amount of from about 15 to about 50% by weight, based on the weight of the second composition, and (b) (vi) is present in an amount of from about 3 to about 12% by weight, based on the weight of the second composition.
